(19) United States
(12) Patent Application Publication
(10) Pub. No.: US 2020/0102602 A1
(43) Pub. Date: Apr. 2, 2020

(54) Diagnostic Assays for Detecting, Quantifying, and/or Tracking Microbes and Other Analytes

(71) Applicants: Locus Agriculture IP Company, LLC, Solon, OH (US); Intelligent Material Solutions Inc., Princeton, NJ (US)

(72) Inventors: Paul ZORNER, Encinitas, CA (US); Eric J. MATHUR, Encinitas, CA (US); Josh COLLINS, Wallingford, PA (US); Howard BELL, Princeton, NJ (US); Scott Alan SHIBATA, Irvine, CA (US)

(21) Appl. No.: 16/614,125

(22) PCT Filed: May 17, 2018
(86) PCT No.: PCT/US2018/033222

§ 371 Date: Nov. 15, 2019

Related U.S. Application Data
(60) Provisional application No. 62/507,895, filed on May 18, 2017.

(51) Int. Cl. C12Q 1/6816; C12Q 1/686; C12Q 1/689; C12Q 1/70

(52) U.S. Cl. CPC C12Q 1/6816; C12Q 1/686; C12Q 2563/155; C12Q 1/701; C12Q 1/689

(57) ABSTRACT

The subject invention provides methods and assays for multiplexed detection of analytes using nanocrystals that are uniform in morphology, size, and composition based on their unique optical characteristics. The described methods and assays are particularly useful for detection of microbes and/or microbe-based agents in a complex environmental sample.

### CROSS-REFERENCE TO RELATED APPLICATION

This application claims the benefit of U.S. provisional application Ser. No. 62/507,895, filed May 18, 2018, which is incorporated herein by reference in its entirety.

### BACKGROUND OF THE INVENTION

Farming, forestry, and other means of producing food, nutritional additives, fiber and natural materials is becoming increasingly difficult due to numerous environmental challenges. Such challenges include pest resistance, extreme temperatures, and pests.

In order to boost yields and protect crops against pathogens, pests, and disease, farmers have relied heavily on the use of synthetic chemicals and chemical fertilizers; however, when overused or improperly applied, these substances can run off into surface water, leach into groundwater, and evaporate into the air. As sources of air and water pollution, these substances are increasingly scrutinized, making their responsible use an ecological and commercial imperative. Even when properly used, the over-dependence and long-term use of certain chemical fertilizers and pesticides deleteriously alters soil ecosystems, reduces stress tolerance, increases pest resistance, and impedes plant and animal growth and vitality.

To empower farmers globally to sustainably grow more food and nutritional supplements as well as foresters to sustainably produce more fiber and structural materials, microorganisms are increasingly utilized. Microbes such as bacteria, yeast and fungi, and their byproducts, are useful in many settings including agriculture, animal husbandry and forestry, and remediation of soils, water and other natural resources.

Farmers are increasingly embracing the use of biological agents such as live microbes, bio-products derived from these microbes, and combinations thereof, for example, as pesticides. These biological agents have important advantages over other conventional pesticides. The advantages include: 1) less harmful compared to conventional chemical pesticides; 2) more efficient and specific; 3) often biodegrade quickly, leading to less environmental pollution.

While enormous potential exists for the use of microbes and microbe-based agents, the ability to detect and/or track such microbes and microbe-based agents in the environment has been limited. The ability to detect or trace the microbes and microbe-based agents would be particularly beneficial for agriculture, including for applications in growing crops, ornamentals, turf, timber, and animals.

Thus, detection of microbes, including pathogens as well as beneficial microbes, or microbe-based agents, in the field would reflect variations in the environment and promote taking appropriate actions to improve plant health. Moreover, detecting and monitoring microbial pathogens in the environment can also be beneficial for promoting human health.

Traditional procedures used for detecting microbes typically involve culturing the specimens and detecting microbial activity. In general, the target microbes are inoculated in a culture medium specific to such target microbes, which provides all the nutrients for their growth. The specimen may be an untreated natural sample, or it may be a sample that has been pre-treated by, for example, membrane filtration.

The detection methods commonly utilize at least one analytical reagent that binds to the specific target and produces a detectable signal. These analytical reagents typically include a probe molecule such as an antibody or oligonucleotide that can bind to the target with a high degree of specificity and affinity, and a detectable label such as a covalently-linked fluorescent dye molecule that can be detected by proper equipment. Typically, the binding properties of the probe molecule define the specificity of the detection method, and the detectability of the associated label determines the sensitivity of the detection method.

Although detection methods with fluorescent dyes possess significant advantages such as high sensitivity, low background, and accurate measurement, and often provide useful results in biomedical research, they are not suitable for detecting and tracking microbes and microbe-based agents for the agriculture industry. Reasons include 1) most common fluorophores are aromatic organic molecules that have both absorption and emission bands located in the UV/visible portion of the spectrum; 2) the lifetime of the fluorescence emission is usually short, on the order of 1 to 100 ns; 3) it is often not possible to integrate a fluorescent signal over a long detection time due to photobleaching; and 4) detection of fluorophores requires sophisticated equipment.

Thus, there remains a need for devices and methods to detect and/or track beneficial microbes, microbe-based agents, and pathogens in the environment quickly and easily, without requiring significant sample preparation steps, to yield accurate diagnostic information.

### SUMMARY OF THE INVENTION

The present invention provides methods and devices to efficiently and accurately detect, quantify and/or track microbes, microbe-based agents, and/or other analytes in environmental and food samples. The samples may be, for example, soil, water, oil, waste, food, foliage, and/or biological samples from livestock or other animals.

The analytes can be microbes, microbe-based agents and/or analytes arising from the presence or activity of microbes. The microbes can be beneficial microorganisms or pathogens, including agricultural pathogens.

In preferred embodiments, the present invention provides in-field diagnostic assays to quickly, efficiently, and accurately detect, quantify, and/or track analytes of interest. Advantageously, multiple analytes can be detected simultaneously. Furthermore, the analytes can be detected at low concentrations, in complex samples, and with negligible, or no, sample preparation.

Advantageously, the assays of the present invention employ tunable nanocrystals as detection labels to identify the presence, and/or quantify, one or more analytes of interest (e.g., beneficial microbes, microbe-based agents, and/or pathogens). This tunability facilitates filtering out background interference, such as from chromophores in a sample. This tunability also makes it possible to detect multiple analytes at the same time. The assay may detect, for example, 1, 2, 3, 4, 5, 10, 15, or 20 or more analytes simultaneously from a single sample.

The nanocrystals are characterized by a uniform morphology and a uniform size. In addition, the nanocrystals can possess their own unique optical and magnetic properties such as optical emission spectral profiles, optical absorption spectral profiles, optical power dependency profile, optical lifetime signatures (rise and decay times), and surface functionality. For example, the nanocrystals may be surface modified to enable them to specifically bind to the analyte(s) of interest. The surface modification may be achieved by, for example, linking the nanocrystals to antibodies, proteins, aptamers, nucleotides, and/or other compounds.

In one embodiment, the nanocrystals are inorganic luminescent or electromagnetically active materials that absorb energy acting upon them and subsequently emit the absorbed energy. In one embodiment, the nanocrystals are stokes (down-converting) phosphors. Phosphors that absorb energy in the form of a photon and emit a lower frequency (lower energy, longer wavelength) band photon are down-converting phosphors.

In another embodiment, the nanocrystals are anti-stokes (up-converting) phosphors. Phosphors that absorb energy in the form of two or more photons in a low frequency and emit in a higher frequency (higher energy, shorter wavelength) band are up-converting phosphors.

In one embodiment, the nanocrystals are rare earth (RE)-containing particles. RE elements include yttrium and the elements of the lanthanide (Ln) series, i.e., lanthanum (La), cerium (Ce), praseodymium (Pr), neodymium (Ne), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

It is advantageous to use nanocrystals with different excitation and/or emission wavelengths, and/or different rise and decay rates, for the detection of more than one analyte in a single assay.

The method can comprise the steps of: providing an environmental or food sample suspected of having an analyte of interest, contacting the sample with a plurality of nanocrystals, and detecting the nanocrystals that bind to the analyte.

Microbes that can be detected, quantified and/or tracked according to the subject invention include, but are not limited to bacteria, archaea, yeast, fungi, viruses, protozoa, and multicellular organisms. The microbe-based agents that can be analytes according to the subjection invention include, but are not limited to, composition containing microbes, microbe metabolites and other microbe growth by-products. In one embodiment, the present invention further provides methods for detecting a product produced by an entity (such as an animal or plant) in response to a microbe and/or microbe-based agent.

Advantageously, the assays of the subject invention can be utilized to facilitate tracking of the analytes in the environment or food chain.

The assays of the subject invention can be used in a wide range of settings including, but not limited to, crops, livestock, forestry, turf management, ornamentals, pastures, aquaculture, waste treatment, the food chain, and animal health.

In specific embodiments, the methods of the present invention comprise a step of applying the sample to a substrate to facilitate performing the analytical assay. The surface of the substrate may have associated therewith, for example, antibodies, proteins, aptamers, nucleotides, and/or other compounds that specifically bind to, or otherwise associate with, the analyte. The assays can utilize, for example, a lateral flow format, multi-well array, or microfluidics.

In a specific embodiment, the subject invention provides a lateral flow or microfluidic assay format where the nanocrystals in the detectable label may be an up-converting phosphor (UCP). In one embodiment, the detection device detects the up-converting emission wavelength. In another embodiment, the detection device detects the phosphor lifetime signature.

The ability to adjust the size, morphology, absorption, emission, rise time, decay time, power density, and other properties of phosphor particles, such as up-converting nanocrystals (UCNC) or submicron phosphor particles, enables the formation of materials with a vast array of distinctive signatures. The versatility of the rare earth UCNC platform significantly increases the ability to have a broad detection capability using a single reader system. Additionally, the ability to optically tune the rare earth nanoparticle or submicron particle unique spectral fingerprints provides highly advantageous multiplexing capabilities.

The methods of the subject invention facilitate rapid, sensitive, and inexpensive, detection and/or quantification of microbes and/or microbe-based agents of interest in complex samples. The use of nanocrystals as labels according to the subject invention provides a rapid, multiplexed and specific assay platform capable of detecting low levels of analyte targets in complex environmental and food samples, such as, for example, in the case of food, agriculture, and livestock samples.

### DETAILED DESCRIPTION OF THE INVENTION

The present invention provides methods and devices to efficiently and accurately detect, quantify, and/or track microbes, microbe-based agents, and/or other analytes. The analytes can be detected in environmental or food samples, such as in soil, water, food, waste, oil, plants and biological samples from animals. The microbes can be, for example, beneficial microorganisms or pathogens, including agricultural pathogens and animal pathogens.

The methods of the present invention employ nanocrystals as detection labels to detect one or more analytes of interest (e.g., beneficial microbes, microbe-based agents, and/or pathogens). Advantageously, multiple analytes can be detected simultaneously in a single assay.

According to the present invention, the nanocrystals exhibit tunable physical properties and, advantageously, have controlled size uniformity, shape selectivity and surface functionality. For example, the nanocrystals may be surface modified to enable them to specifically bind to an analyte of interest. The surface modification may be achieved by linking the nanocrystals to, for example, antibodies, proteins, aptamers, nucleotides, and/or other compounds.

In one embodiment, the present invention provides methods for detecting an analyte in a sample comprising the steps of:

contacting the sample with a plurality of nanocrystals, wherein the nanocrystals have been surface modified with an entity that specifically binds to the target analyte,

separating the nanocrystals bound to the analyte from unbound nanocrystals, and

detecting the nanocrystals that bind to the analyte.

The microbes detected, quantified and/or tracked according to the subject invention can be any prokaryotic or eukaryotic microscopic organism, including, but not limited to bacteria (e.g., spore or vegetative, Gram positive or Gram negative), archaea, yeast, fungi (e.g., filamentous fungi and fungal spores), viruses, protozoa, or multicellular organisms. In some cases, the microorganisms of particular interest are those that are pathogenic. The term “pathogen” is used to refer to any pathogenic microorganism. In other instances the microbe is beneficial.

In a specific embodiment, the method is used to detect, optionally in a complex environmental sample, pathogens that cause citrus greening disease. Citrus greening disease also known as Huanglongbing (HLB) is caused by the phloem-limited fastidious prokaryotic α-proteobacterium Candidatus Liberibacter spp., Ca. africanus, and Ca. L. americanus.

The methods described herein are suitable for use on any tree or other plant that is infected or may be infected with citrus greening disease. Exemplary plants include, but are not limited to, any cultivar from the genus Citrus, including but not limited to Citrus sinensis (navel oranges), lemon (C. limon), lime (C. latifolia) grapefruit (C. paradise), sour orange (C. aurantium), and mandarin (C. reticulata).

In other specific embodiments, the assays of the subject invention are used to detect, quantify and/or track the plant pathogens that cause Potato Late Blight, Grape Powdery Mildew, Red Blotch, Tobacco Mosaic Virus, Fire blight and/or Pierce's Disease.

The sample can be, but is not limited to, water, soil, food, plant, air, waste, biological samples from animals, dust, and samples collected from surfaces.

Collection may be achieved by any of a variety of methods, including, but not limited to, use of a sponge, wipe, swab (e.g., a wound fiber product), film, brush (e.g., having rigid or deformable bristles), and the like, and combinations thereof.

In one embodiment, the analyte is a microbe-based agent. Microbe-based agents according to the subjection invention include, but are not limited to, composition that contain microbes, microbe metabolites and other microbe growth by-products. In specific embodiments, the microbe-based agent is a microbial biosurfactant or mycotoxin.

The assays of the subject invention can be utilized to facilitate tracking of microbes, microbe-based agents, and other analytes in the environment or food chain.

In preferred embodiments, the nanocrystals are monodisperse particles in crystalline form having a rare earth-containing lattice, uniform three-dimensional size, and uniform polyhedral morphology. Preferably, the monodisperse particles are capable of self-assembly into superlattices due to their uniform size and shape.

In one embodiment, the nanocrystals are inorganic luminescent or electromagnetically active materials that absorb energy acting upon them and subsequently emit the absorbed energy. Such nanocrystals can act as phosphors that continue to emit light for greater than 10⁻⁸ seconds after the removal of the absorbed light. The half-life of the afterglow, or phosphorescence, of a phosphor typically ranges from about 10⁻⁶ seconds to days.

In certain embodiments, the nanocrystals according to the subject invention are stokes (down-converting) phosphors. Phosphors that absorb energy in the form of a photon and emit a lower frequency (lower energy, longer wavelength) band photon are down-converting phosphors.

In other embodiments, the nanocrystals are anti-stokes (up-converting) phosphors. Phosphors that absorb energy in the form of two or more photons in a low frequency and emit in a higher frequency (higher energy, shorter wavelength) band are up-converting phosphors. Up-converting phosphors can be, for example, irradiated by near infra-red light, a lower energy, longer wavelength light, and emit visible light that is of higher energy and a shorter wavelength.

In one embodiment, the nanocrystals are rare earth (RE)-containing particles. RE elements include yttrium and the elements of the lanthanide (Ln) series, i.e., lanthanum (La), cerium (Ce), praseodymium (Pr), neodymium (Ne), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

In certain embodiments, the down-converting nanocrystals of the invention can be excited at a wavelength between 1 nm and 400 nm, preferably, between 10 nm and 400 nm.

In other embodiments, the up-converting nanocrystals of the invention can be excited at a wavelength between 700 nm and 2000 nm, preferably, between 800 nm and 1500 nm, more preferably, 900 nm and 1000 nm. In a specific embodiment, the up-converting nanocrystals can be excited at a wavelength from 960 nm to 980 nm.

In one embodiment, the nanocrystals of the invention emit light at a wavelength from 400 nm to 12,000 nm.

In one embodiment, the nanocrystals used in the present assay may be combined with a second reporter such as quantum dots, carbon nanotubes, as well as magnetic and dye-doped nanoparticles. Combining nanocrystals with other waveshifting and absorbing materials allows for additional multiplexing and functionality. Two complimentary particles such as an upconverting nanocrystal and a downconverting quantum dot that absorbs the emission of the upconverting nanocrystal with the same capture antibodies will bind to a target. When activated with a 980 nm light the quantum dot by itself does not emit but when in proximity of a upconverting nanocrystal, the nanocrystal will transfer the necessary energy to activate the quantum dot. The only time the two particles are close enough is if they bind to a specific target. In a microfluidic system, binding effects can be quantified in real time.

Adding magnetic properties to the nanocrystals allows for faster processing time before analysis as the particles can be funneled into the assay with a magnet. The magnetic properties can also be read during detection. Rare-Earth crystals combined with other metals exibit different properties such as paramagnetic and ferromagnetic.

Organic dyes coated over the nanocrystals form a filter and can benefit spectral interference. Lanthanide lines sometimes overlap and adding organic materials allows for blocking of certain regions in the spectrum to produce single emissions.

Multiple nanocrystals possessing distinct sizes, lifetimes and/or morphologies can be combined and introduced into or onto a complex environmental sample providing multiple unique detectable labels that can be used for multiple analyte detections. The rare earth nanocrystals are advantagous because of their relatively long phosphorescence lifetime decays attributed to, for example, the trivalent rare earth (or lanthanide) metals.

It is advantageous to use nanocrystals with different excitation and/or emission wavelengths for the detection of more than one analyte in a single assay by using different labels to identify particular targets. For example, it is possible to generate multiple spectrally-separate colors (e.g., blue, green, and red) by means of infrared (IR), ultra violet (UV), or electron excitation to measure phosphor emission wavelengths, intensity amplitudes, and the number of analytes at the same time. In particular, the immunocytochemical use of nanocrystal conjugates with capture molecules allows a sensitive detection of small quantities of analyte in the environmental samples.

Advantageously, the multiplexing property of the assay using nanocrystals makes it possible to detect an analyte of interest in a complex environmental or food sample without interference from sample components. For example, nanocrystals with tunable characteristic allow the quantification of analytes of interest from interfering chromophores that are present in soil or plant samples.

In one embodiment, the subject invention also provides a method for the preparation of the nanocrystals. The method employs the steps of: in a reaction vessel, dissolving at least one precursor metal salt in a solvent to form a solution; placing the reaction vessel in a heated salt bath having a temperature of at least about 340° C.; applying heat to the salt bath to rapidly decompose the precursor metal salts in the solution to form the monodisperse particles; keeping the reaction vessel in the salt bath for a time sufficient to increase the size of the monodisperse particles; removing the reaction vessel from the salt bath; and quenching the reaction with ambient temperature solvent.

Advantageously, the present invention provides a sensitive assay with a detection sensitivity for microbe at 10³ CFU/mL and lower. In preferred embodiments the sensitivity is 10² CFU/mL, more preferably, 10¹ CFU/mL. Thus, the assay can detect microbes in a complex sample ranging from 10¹ CFU/mL to 10⁹ CFU/mL and higher.

The present invention also provides a sensitive assay with a detection sensitivity for microbe-based agents as low as 0.001 ng/mL.

Advantageously, the assays can be performed in the field. In certain embodiments, the assays are performed within 1000, 500, 250, 100, 50, 20, 10, 5 or even 1 yard or less from wherein the sample was obtained. Further, the assay may be performed, for example, within 60, 45, 30, 20, 10, 5, or even 1 minute or less from when the sample was taken.

In one embodiment, the methods can be used for simultaneously detecting one or more analytes in a complex environmental sample. The detection can be accomplished in 60 minutes or less, 50 minutes or less, 40 minutes or less, 30 minutes or less, 20 minutes or less, 10 minutes or less, or 5 minutes or less. In preferred embodiments, the assay is conducted more quickly and/or with less sample preparation than assays utilizing PCR or standard ELISA. The results may be read immediately upon completion of the assay and/or stored and/or transmitted to another location. For example, the results may be transmitted electronically for storage and/or further analysis. The results may be, for example, transmitted to an electronic storage cloud or other stored database.

These tools can be used to conduct quality control and assess product specifications both immediately following production as well as at a farmer's field just prior to application. This facilitates rapid product release that is highly beneficial in a local microbial fermentation system, as well as in any system, because it is faster, cheaper, and more accurate than other current methods.

The assays of the subject invention can also be used to confirm the characteristics of a microbial product purchased by a consumer. This aspect of the invention has great value as many biologicals lose potency over time and become well below stated potency by the time they are bought or used. This aspect also helps to manage inventory, and determine which products are off specification for products with single microbes or those that contain several.

A plant's nutrition, growth, and proper functioning are dependent on the quantity and distribution of robust populations of natural microflora that, in turn, are influenced by soil fertility, tillage, moisture, temperature, aeration, organic matter, and many other factors. Prolonged drought, variable rainfall, and other environmental variations, including the proliferation of nematodes and other pests, influence those factors and affect soil diversity and plant health. These environmental variables manifest themselves in multiple dimensions, including geography, seasonality in a given year, and differences between years. They also exist within a specific farm and even within as small an area as an acre, or less or between animal species or even individual animals within a species. Using the assays of the subject invention to analyze, quickly and accurately, microbial (beneficial and pathogenic) presence and ecology within meta and micro environments provides much greater power to farmers, regulatory officials, compliance officials, basic producers, distribution agents in the supply chain and other organizations or individuals wishing to better enhance their assets, manage pathogens, and optimize the efficiency and economic performance of their business.

### Nanocrystals

The nanocrystals, useful according to the subject invention, are inorganic luminescent or electromagnetically active materials that absorb energy acting upon them and subsequently emit the absorbed energy. Such nanocrystals can act as phosphors that continue to emit light for greater than 10⁻⁸ seconds after the removal of the absorbed light. The half-life of the afterglow, or phosphorescence, of a phosphor typically ranges from about 10⁻⁶ seconds to days.

The nanocrystals of the invention may have different optical properties based on their composition, their size, and/or their morphology (or shape). In one embodiment, the invention relates to a combination of at least two types of nanocrystals, where each type is a plurality of monodisperse particles having a single pure crystalline phase of a rare earth-containing lattice, a uniform three-dimensional size, and a uniform polyhedral morphology; and where the types of monodisperse particles differ from one another by composition, by size, or by morphology. In a preferred embodiment, the types of monodisperse particles have the same composition but different morphologies.

In one embodiment, the nanocrystals according to the subject invention are stokes (down-converting) phosphors. Phosphors that absorb energy in the form of a photon and emit a lower frequency (lower energy, longer wavelength) band photon are down-converting phosphors.

In another embodiment, the nanocrystals are anti-stokes (up-converting) phosphors. Phosphors that absorb energy in the form of two or more photons in a low frequency and emit in a higher frequency (higher energy, shorter wavelength) band are up-converting phosphors. Up-converting phosphors, for example, are irradiated by near infra-red light, a lower energy, longer wavelength light, and emit visible light which is of higher energy and a shorter wavelength.

In one embodiment, the nanocrystals are rare earth (RE)-containing particles. RE elements include yttrium and the elements of the lanthanide (Ln) series, i.e., lanthanum (La), cerium (Ce), praseodymium (Pr), neodymium (Ne), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

In a specific embodiment, the nanocrystals of the invention have a rare earth-containing lattice that may be an yttrium-containing lattice or a lanthanide-containing lattice. The lattice contains yttrium (Y) or a lanthanide (Ln) in its +3 oxidation state. The charge is balanced in the lattice by the presence of an anion such as a halide (fluoride, F, being preferred), an oxide, an oxysulfide, an oxyhalide (e.g., OCl), a sulfide, etc. Alkali metals, i.e., lithium (Li), sodium (Na), potassium (K), rubidium (Rb), and cesium (Cs) and/or alkali earth metals beryllium (Be), magnesium (Mg) calcium (Ca), strontium (Sr), and barium (Ba) may also be a component of the host lattice. The alkali metals or alkaline earth metals are often called “lattice modifiers.”

The nanocrystals may vary in size. In one embodiment, crystals of the invention may be described as nanocrystals with their largest dimension ranging approximately 1 nm to 1,000 nm in size, preferably, from 5 nm to 750 nm, more preferably, from 10 nm to 500 nm, most preferably, 20 nm to 400 nm. Large crystals, with at least one dimension of approximately 1 μm to 400 μm, represent another embodiment of the invention. The size of the crystal depends on the stoichiometric ratio of elements making the crystal or the stoichiometric ratio precursor used to prepare the particle as well as the length of reaction time.

Nanocrystals used according to the subject invention preferably have a single pure crystalline phase of a RE-containing lattice. In one embodiment, the nanocrystal is a α, β, or cubic-phase crystal. In a preferred embodiment, the nanocrystal is a hexagonal (β)-phase particle.

For the synthesis of monodisperse particles of the invention, the alkali metal or alkaline earth metal present in the lattice may determine the crystal symmetry providing morphological control over the particles as well as independent tunability of a particle's other properties, such as the optical properties of a luminescent particle. For example, the crystal symmetry of LiYF₄, NaYF₄, and KYF₄ are tetragonal, hexagonal, and trigonal, respectively.

The chemical composition of the particles of the invention provides unique polyhedral morphologies. Representative yttrium-containing lattices include, but are not limited to LiYF₄, BaYF₅, BaY₂F₈NaYF₄, KYF₄, Y₂O₂S, Y₂O₃, and the like. The lanthanide-containing lattice may be one having any element of the lanthanide series. Representative lanthanide-containing lattices include, but are not limited to, LaF₃, CeF₃, PrF₃, NeF₃, PmF₃, SmF₃, EuF₃, GdF₃, TbF₃, DyF₃, HoF₃, ErF₃, TmF₃, YbF₃LuF₃, NaGdF₄, Gd₂OS₃, LiHoF₄, LiErF₄, CeO, SrS, CaS, GdOCl, and the like.

In one embodiment, the chemical composition of the particles may contain dopants and lattice modifiers, which impart unique properties to the composition.

The morphology of the nanocrystals can be spherical, hexagonal, cubic, rod-shaped, diamond-shaped, odd shape such as a mushroom or a dumbbell. Advantageously, UCNC do not photobleach and allow high power density excitation over long term exposure with simultaneous signal integration. They can be stored indefinitely without a decrease in light emitting efficiency and thus they allow repeated irradiation and analysis. Unlike previous inorganic markers of the past, the nanocrystals are uniform and provide a consistent signal based upon their concentration. If the crystals are amorphous the distribution of the atoms is not consistent, there are defects in the structures and the emitted optical signal cannot be quantified. The invention takes advantage of the uniform morphology of the crystals. Similar to a remote control, an infrared pulsed light is emitted from the crystal during the test. Such properties facilitate the quantification of analytes of interest in a complex environmental sample.

### A. Down-Converting Phosphors

Down-converting phosphor materials include RE element doped oxides, RE element doped oxysulfides, RE element doped fluorides. Examples of down-converting phosphors include, but are not limited to Y₂O₃:Gd, Y₂O₃:Dy, Y₂O₃:Tb, Y₂O₃:Ho, Y₂O₃:Er, Y₂O₃:Tm, Gd₂O₃:Eu, Y₂O₂S:Pr, Y₂O₂S:Sm, Y₂O₂S:Eu, Y₂O₂S:Tb, Y₂O₂S:Ho, Y₂O₂S:Er, Y₂O₂S:Dy, Y₂O₂S:Tm, Y₂O₂S:Eu (red), Y₂O₃:Eu (red), and YV0₄:Eu (red). Other examples of down-converting phosphors are sodium gadolinium fluorides doped with other lanthanides, e.g., NaGdF₄:Tb, wherein the Tb can be replaced with Eu, Dy, Pr, Ce, etc. Lanthanide fluorides are also known as down-converting fluorides, e.g., TbF₃, EuF₃, PrF₃, and DyF₃.

### B. Up-Converting Phosphors

Up-converting phosphors derived from RE-containing host lattices, such as described above, doped with at least one activator couple comprising a sensitizer (also known as an absorber) and an emitter. Suitable up-converting phosphor host lattices include: sodium yttrium fluoride (NaYF₄), lanthanum fluoride (LaF₃), lanthanum oxysulfide, RE oxysulfide(RE₂O₂S), RE oxyfluoride (RE₄O₃F₆), RE oxychloride (REOCl), yttrium fluoride (YF₃), yttrium gallate, gadolinium fluoride (GdF₃), barium yttrium fluoride (BaYF₅, BaY₂F₈), and gadolinium oxysulfide, wherein the RE can be Y, Gd, La, or other lanthanide elements. Suitable activator couples are selected from: ytterbium/erbium, ytterbium/thulium, and ytterbium/holmium. Other activator couples suitable for up-conversion may also be used.

By combination of RE-containing host lattices with just these three activator couples, at least three phosphors with at least three different emission spectra (red, green, and blue visible light) are provided. Generally, the absorber is ytterbium and the emitting center can be selected from: erbium, holmium, terbium, and thulium; however, other up-converting phosphor particles of the invention may contain other absorbers and/or emitters. The molar ratio of absorber:emitting center is typically at least about 1:1, more usually at least about 3:1 to 5:1, preferably at least about 8:1 to 10:1, more preferably at least about 11:1 to 20:1, and typically less than about 250:1, usually less than about 100:1, and more usually less than about 50:1 to 25:1, although various ratios may be selected by the practitioner on the basis of desired characteristics (e.g., chemical properties, manufacturing efficiency, excitation and emission wavelengths, quantum efficiency, or other considerations). For example, increasing the Yb concentration slightly alters the absorption properties, which is useful for biomedical applications. Additionally, the introduction of other rare earth and transition metal dopants, alterations in the doping concentrations, and host lattice modifications, all provide further tunability over spectral profiles as well as rise and decay times.

The optimum ratio of absorber (e.g., ytterbium) to the emitting center (e.g., erbium, thulium, or holmium) varies, depending upon the specific absorber/emitter couple and desired spectral profile and lifetime. For example, the absorber:emitter ratio for Yb:Er couples is typically in the range of about 1:1 to about 100:1, whereas the absorber:emitter ratio for Yb:Tm and Yb:Ho couples is typically in the range of about 500:1 to about 2000:1. These different ratios are attributable to the different matching energy levels of the Er, Tm, or Ho with respect to the Yb level in the crystal. For most applications, up-converting phosphors may conveniently comprise about 10-30% Yb and either: about 1-2% Er, about 0.1-0.05% Ho, or about 0.1-0.05% Tm for optimal quantum efficiency, although other formulations may be employed.

In some embodiments, inorganic phosphors are optimally excited by infrared radiation of about 900 to 1000 nm, preferably about 960 to 980 nm. For example, but not by limitation, a microcrystalline inorganic phosphor of the formula YF₃:Yb_{0.10}Er_{0.01}exhibits a luminescence intensity maximum at an excitation wavelength of about 980 nm. Up-converting phosphors of the invention typically have emission maxima that are in the visible to near infrared range. For example, specific activator couples have characteristic emission spectra: ytterbium-erbium couples have emission maxima in the red (660 nm) or green (540 nm) portions of the visible spectrum, depending upon the phosphor host; ytterbium-holmium (535 nm) couples generally emit maximally in the green portion, ytterbium-thulium typically have an emission maximum in the blue (480 nm), red (635 nm) and infrared (800 nm) range, and ytterbium-terbium usually emit maximally in the green (545 nm) range. For example, Y_{0.80} Yb_{0.19} Er_{0.01}F, emits maximally in the green portion of the spectrum.

The phosphor particle of the invention can be excited at 915 nm instead of 980 nm where the water absorption is much higher and more tissue heating occurs. The ratio(s) chosen will generally also depend upon the particular absorber-emitter couple(s) selected, and can be calculated from reference values in accordance with the desired characteristics. It is also possible to control particle morphologies by changing the ratio of the activators without the emission properties changing drastically for most of the ratios but quenching may occur at some point.

### C. Particle Properties Based on Composition, Morphology, and Size

Properties of the monodisperse particles can be tuned in a variety of ways. The properties of the monodisperse particles, the characteristic absorption and emission spectra, may be tuned by adjusting their composition, e.g., by selecting a host lattice, and/or by doping. Advantageously, given their uniform polyhedral morphology, the monodisperse particles exhibit anisotropic properties. Particles of the same composition but different shape exhibit different optical properties due to their shape and/or size.

In one embodiment, the monodisperse particles are varied in composition and/or shape to give different decay lifetimes. Having different spectral decay lifetimes allows unique phosphor particles to be differentiated from one another. The ability to have monodisperse particles of the same composition but different morphologies according to the invention permits use of one composition (especially in regulated industries such as pharmaceuticals or medical devices) but to distinguish its morphologies through their unique optical properties.

Thus, in addition to the characteristic absorption and emission spectra that can be obtained the rise and decay times of a monodisperse particle of the invention can also be tuned by particle size and morphology. The rise time is measured from the moment the first excitation photon is absorbed to when the first emission photon is observed. The decay time is measured by the slope of the emission decay, or the time it takes for the phosphor to stop emitting once the excitation source is turned off. This is also described as the time it takes for depletion of electrons from the excited energy levels. By changing the dopant ratio, the rise and decay times can be reliably altered.

Typically, an excited state population decays exponentially after turning off the excitation pulse by first-order kinetics, following the decay law, I(t)=I₀ exp (−t/τ), whereby for a single exponential decay I(t)=time dependent intensity, I₀=the intensity at time 0 (or amplitude), and τ=the average time a phosphor (or fluorophor) remains in the excited state (or <t>) and is equal to the lifetime. (The lifetime τ is the inverse of the total decay rate, τ(T+kₙᵣ)⁻¹, where at time t following excitation, T is the emissive rate and kₙᵣ is the non-radiative decay rate). In general, the inverse of the lifetime is the sum of the rates which depopulate the excited state. The luminescence lifetime can be simply determined from the slope of a plot of lnl(t) versus t (equal to l/τ). It can also be the time needed for the intensity to decrease to lie of its original value (time 0). Thus, for any given known emission wavelength, a number of parameters fitting the exponential decay law can be monitored to identify a particular phosphor or group of phosphors, thus permitting their use, for example, in developing unique anti-counterfeiting codes, signatures, or labels/taggants.

In most instances, lifetimes are controlled by variations in the crystal composition or overall particle size. However, by controlling the particle morphology and uniformity as with the monodisperse particles of the invention one can create particles of visually distinct morphologies possessing lifetimes that are unique to that morphology while maintaining identical chemical compositions among the various morphologies. This feature allows for a highly complex optical signature or taggant which, may be used in serialization and multiplexing assays or analysis in various fields such as, for example, assays, biomedical, optical computing, as well as use in security and authentication.

Particle size and morphology may be controlled by varying reaction conditions such as stoichiometric precursor metal salt ratio, heating rate of the salt bath, and reaction time. The initial rate of heating in the salt bath is important in determining the morphology by selecting which crystal planes will undergo the most rapid growth. Final particle size is determined by total reaction time in the salt bath as well as precursor ratios. After the reaction vessel reaches the temperature of the salt bath, the longer the time the vessel remains in the salt bath the larger the particles may grow.

### D. Superlattice Assembly

Due to their uniformity in size and morphology, the monodisperse particles of the invention are able to self-assemble into superlattice structures. These superlattice structures represent the lowest free energy conformation for the assemblage. This uniform build-up is accomplished with monodisperse particles of uniform size and morphology as according to the invention. The superlattices form via interfacial self-assembly, building hierarchical structures with orders on different length scales.

Superlattices of the monodisperse particles of the invention may be formed by suspending the particles in a solvent and then drop-casting them onto a surface. As the solvent slowly evaporates, the particles arrange themselves into a superlattice with both positional and orientational order. Any solvent which disperses the particles may be used, such as, but not limited to, benzene, carbon tetrachloride, chlorobenzene, chloroform, cyclohexane, dimethyl-formamide, dimethyl sulfoxide, ethanol, heptane, hexane, pentane, tetrahydrofuran, toluene, with nonpolar organic solvents such as hexane being preferred.

Superlattices of the invention may be transparent films of the monodisperse particles of the invention, particularly with monodisperse nanoparticles of the invention. In order to form a superlattice the constituent particles must be of identical or nearly identical size and shape. When both conditions are met a uniform, patterned, monolayer of particles forms. Advantageously, the monodisperse particles of the invention meet these criteria for uniform size and uniform morphology. Due to the small size and uniformity of the particles of the invention, there is no scattering of light and as a result a transparent film is obtained.

### Functionalization of the Nanocrystals

In one embodiment, the nanocrystals have been functionalized with one or more capture molecules. This can be done by, for example, linking the nanocrystals to antibodies, proteins, polypeptides, aptamers, nucleotides, and/or other compounds that specifically bind to an analyte such as a target microbe or a microbe-based agent. In another embodiment, the analyte target could also be any of a range of host biomolecules induced to express in response to infection by a pathogenic microorganism.

“Specific,” as used herein, refers to an antibody, or other entity, that only recognizes the target to which it is specific or that has significantly higher binding affinity to the target to which it is specific compared to binding to molecules to which it is non-specific. The binding affinity measures the strength of the interaction between an epitope and an antibodies antigen binding site. Higher affinity antibodies will bind a greater amount of antigen in a shorter period of time than low-affinity antibodies. Thus, the binding affinity constant can vary widely from below 10⁵ mol⁻¹ to above 10¹² mol⁻¹.

In a preferred embodiment, the antibody may comprise a complete antibody molecule having full length heavy and light chains or a fragment thereof and may be, but are not limited to, Fab, modified Fab, Fab′, modified Fab′, F(ab′)2, Fv, single domain antibodies (e.g., VH or VL or VHH), scFv, bi, tri or tetra-valent antibodies, Bis-scFv, diabodies, triabodies, tetrabodies and epitope-binding fragments of any of the above (see, for example, Holliger and Hudson, 2005, Nature Biotech. 23(9):1126-1136; Adair and Lawson, 2005, Drug Design Reviews—Online 2(3), 209-217). The antibodies can be specific to, for example, proteins, or epitopes of proteins, that is expressed at the surface of the target microbes.

Antibodies, including chimeric antibodies, can be used according to the subject invention. Chimeric antibodies are those antibodies encoded by immunoglobulin genes that have been genetically engineered so that the light and heavy chain genes are composed of immunoglobulin gene segments belonging to different species. These chimeric antibodies can be less antigenic. Multi-valent antibodies may comprise multiple specificities or may be monospecific.

The antibodies for use in the present invention can be purchased or they can be generated using various methods, including phage display methods, known in the art. Also, mice, or other organisms, including other mammals, may be used to express antibodies.

The antibody can be of any class (e.g., IgG, IgE, IgM, IgD and IgA) or subclass of immunoglobulin molecule. In one embodiment the antibody for use in the present invention is of the IgG class and may be selected from any of the IgG subclasses IgG1, IgG2, IgG3 or IgG4.

The antibody for use in the present invention may include one or more mutations to alter the activity of the antibody.

Examples of antigens include, but are not limited to, cell surface molecules that are stable or transient plasma membrane components, including peripheral, extrinsic, secretory, integral or transmembrane molecules. In some embodiments, the molecule is exposed at the exterior of the plasma membrane of the cell. In other embodiments, the antigenic determinant is not surface exposed but is instead exposed upon, for example, cell lysis. In certain embodiments, the antigen is a molecule of known structure and having a known or described function, including but not limited to glycoproteins, lipoproteins, and cell wall anchored proteins; the epitope of the antigen may also be a non-protein based biomolecule

In another embodiment, the surface antigen and/or the epitope of the surface antigen may be selected based on genome sequence information. The identification of antigens may involve biological software known in the art (see, for example, Bioinformatics Approach for Cell Surface Antigen Search of Helicobacter pylori, Ragini Tiwari et al., Journal of Pharmacy Research 2012, 5(11), 5184-5187) based on the sequence information of specific motif of interest. For example, programs like SignalP, LipoP, PSORTb, and TMHMMS can be used to filter and select an antigen of interest.

Specifically, SignalP 4.1 server predicts the presence and location of signal peptide cleavage sites in amino acid sequences from different organisms: Gram-positive prokaryotes, Gram-negative prokaryotes, and eukaryotes. The method incorporates a prediction of cleavage sites and a signal peptide/non-signal peptide prediction based on a combination of several artificial neural networks. The website address is: www.cbs.dtu.dk/services/SignalP.

The TMHMM server predicted membrane spanning helices in proteins by searching hydrophobic amino acids. The algorithm predicted number of helices and highlighted spanning the length of the peptides. The web address is: www.cbs.dtu.dk/services/TMHMM. The lipoP server predicted lipoproteins by available lipoprotein signal peptides. The web address is: www.cbs.dtu.dk/services/LipoP.

PSORTb predicts the localization site and the associated probability. Subcellular localization of proteins has been done based on amino acid sequence information. A protein subcellular localization was influenced by several features present within the protein's primary structure, such as the presence of a signal peptide or membrane-spanning alpha-helices. The web address is: http://www.psort.org/psortb/.

Advantageously, the methods of the subject invention can be used to detect microbes that are difficult or impossible to grow in culture, or that can be grown in culture but only very slowly. Because the methods of the subject invention can detect very low numbers of microbes, it is not necessary to grow the microbes from a sample in a culture to increase their numbers prior to performing the assay of the subject invention. Accordingly, the assay of the subject invention can be used to detect microbes that not amendable for cultivarion under standard laboratory conditions, or in culture take longer than 1, 2, 5, 10, 24, 72 or more hours to double in number, or which cannot be grown at all in culture. Thus, the assay of the subject invention can be used to detect, quantify and/or track beneficial microbes such as pasteuria, as well as the pathogens that cause citrus greening disease and zebra chip disease. Viruses can also be detected.

The capture molecules for such difficult-to-culture microbes can be based on antigens identified as described above, as well as through metagenome sequencing. Metagenomics is the study of genetic material recovered directly from environmental samples. Conventional sequencing requires a culture of identical cells as a source of DNA. However, many microorganisms in environmental samples cannot be cultured and thus cannot be sequenced. Advances in bioinformatics, refinements of DNA amplification, and increases in computational power have greatly aided the analysis of DNA sequences recovered from environmental samples, allowing the adaptation of shotgun sequencing to metagenomic samples. The random nature of shotgun sequencing ensures that many of these organisms, which would otherwise go unnoticed using traditional culturing techniques, will be represented by at least some sequence segments.

The genomes of pathogenic microorganisms often contain pathogenicity islands acquired through horizontal gene transfer. These gene islands are incorporated into the genome of pathogenic organisms, but are typically absent from non-pathogenic related species. Pathogenicity island DNA sequences often code for virulence factors which are excellent targets for specific antibodies. These pathogenicity island sequences can be identified via bioinformatic analysis, subcloned, expressed and used as pure antigen for generating antibodies.

A first step of metagenomic data analysis often entails the execution of certain pre-filtering steps, including the removal of redundant, low-quality sequences and sequences of probable eukaryotic origin. Next, metagenomic analysis typically use two approaches in the annotation of coding regions in the assembled contigs. The first approach is to identify genes based upon homology with genes that are already publicly available in sequence databases, by simple BLAST searches. The second, ab initio, uses intrinsic features of the sequence to predict coding regions based upon gene training sets from related organisms. This is the approach taken by programs such as GeneMark and GLIMMER. This approach facilitates the detection of coding regions that lack homologs in the sequence databases.

Metagenomic sequencing is particularly useful in the study of viral communities. As viruses lack a shared universal phylogenetic marker (as 16S RNA for bacteria and archaea, and 18S RNA for eukarya), the only way to access the genetic diversity of the viral community from an environmental sample is through metagenomics.

In accordance with the subject invention, metagenome sequencing can be performed, for example, on a leaf sample having a complex mixture of microbes. Metagenome sequencing can be used to identify DNA coding sequences that can then be cloned and engineered to express peptides and/or full proteins that can then be used to generate antibodies for use in lateral flow assays (or other assays) for detecting, quantifying and/or tracking an uncultureable microbe.

In one embodiment, the surface of nanocrystals may be coated with a surface modifier, for example, polymers such as polyacrylic acid and copolymers such as maleic acid/polyacrylic acid and block copolymers, or an inert silica layer to allow or improve the conjugation of the capture molecule to the particle surface. The nanocrystals conjugated to each type of capture molecule have unique and uniform morphology, size, and/or composition, producing a unique optical lifetime signature.

In one embodiment, the conjugation of the capture molecule is achieved using a method known in the art. Generally, conjugation is accomplished using a carboxylic acid activating reagent for coupling to nuclephiles. In a specific embodiment, the conjugation of the capture molecules is achieved via the N-hydroxysuccinimide (NHS) and/or Sulfo-NHS for preparing amine-reactive esters of carboxylate groups for chemical labeling, crosslinking and solid-phase immobilization. In additional to NHS esters and thiols, imidoesters can also be used as amine-specific functional groups that are incorporated into reagents for protein crosslinking and labeling.

### Assay Formats

In specific embodiments, the methods of the present invention comprise a step whereby target microbes and/or microbe-based agents in an environmental sample become affixed to, or otherwise associated with, a substrate. This step can be accomplished by, for example, treating the surface of a substrate with capture molecules, for example, antibodies, proteins, nucleotides, and other compounds that specifically recognize the target microbe and/or the microbe-based agent. The capture molecules may be the same or different molecules used for functionalizing the surface of nanocrystals to specifically capture the target of interest.

A separating step according to the subject invention may be achieved through methods known in the art. The separation method may involve, but is not limited to, wash, perfusion, and dialysis.

Although not generally necessary, in certain embodiments of the subject invention enrichment techniques such as the use of paramagnetic UCNCs can be used to enrich the sample, thereby further enhancing sensitivity and/or selectivity.

### A. Lateral Flow Assays

In a preferred embodiment, the present invention employs a lateral flow assay, which is utilized to test for the presence, absence, and/or quantity of an analyte of interest in a sample. In one embodiment a “sandwich” assay is used whereby an antibody (or other binding liquid) is immobilized on a solid support to capture a target analyte thereby facilitating the detection and/or quantification by observing bound analyte.

In one embodiment, the assay of the invention is performed on a lateral flow test strip. Lateral flow test strips have a solid support on which the sample-receiving area and the target capture zone(s) are located. The solid support also provides for capillary flow of sample out from the sample receiving area to the target capture zone(s) when the lateral flow test strip is exposed to an appropriate carrier liquid of the sample. The materials of such solid support can be, for example, organic or inorganic polymers, and natural and synthetic polymers. More specific examples of suitable solid supports include, but are not limited to, glass fiber, cellulose, nylon, crosslinked dextran, various chromatographic papers, Diomat™ and nitrocellulose. In a preferred embodiment, the material of the solid support is nitrocellulose. In a further embodiment, the lateral flow test strips may contain one or more target capture zones.

In one embodiment, the lateral flow test strips are constructed for use with a device that directs a particular wavelength of light, for example, infrared, visible, UV light, or with an electron beam, and in turn captures the return wavelength emitted by the nanocrystals when stimulated. Such device is preferably in a handheld form.

In a further embodiment, the subject invention provides a highly sensitive, specific, and quantitative-capable diagnostic platform utilizing a lateral flow assay with the rare earth nanocrystals bound to oligonucleotides or antibodies capable of being read with, for example, a cell phone camera. The assay does not require DNA amplification and can be applied to detect a wide range of agricultural pathogens. In a specific example, the assay can be used to detect Xanthomonas axonopodis pv. manihotis (bacterial blight) in cassava.

Detection methods of agricultural diseases historically require laboratory analysis, limiting their use in resource-limited settings. Traditional lateral flow assays, while easier to utilize in field settings are typically less sensitive than lab-based methods, such as PCR. When an optical reader is combined with a lateral flow assay a several orders of magnitude improvement is achieved over visual reading; however, optical readers are cost prohibitive for distributed use.

In one embodiment, the assay of the subject invention addresses this problem by utilizing nanocrystals conjugated to oligonucleotides, which are then utilized in a lateral flow assay format. The high efficiency and sensitivity of the nanocrystal eliminates the need for a DNA amplification step and the use of an optical reader. Rather, the reader can utilize non-complex technology such as an LED flash and a camera. The flash and the camera can be, for example, those which are typically incorporated into a standard cell phone.

Advantageously, recording the results through a cell phone (or similar device) facilitates the transfer and aggregation of data. This can be used to create a more balanced dataset, from which, for example, machine learning can be applied to better predict outbreaks of agricultural diseases.

In a specific embodiment, the subject invention, provides a lateral flow assay format where the nanocrystals in the detectable label constitute an up-converting phosphor reporter. The consecutive flow technique allows for the use of a reporter such as nanocrystals covered with capture molecules. In certain embodiments, the flow rate can be faster and flow time shorter compared to conventional assays.

The solid support provides for the capillary flow of sample out from the sample receiving area to the target capture zones when the lateral flow test strip is exposed to an appropriate carrier liquid of the sample.

In one embodiment, the lateral flow test strips or microfluidic devices may contain one or more sample receiving areas/channels, which allows the application of multiple samples. Each of the samples may contain a different analyte, or may contain the same analyte. In another embodiment, the sample receiving area comprises the absorbent pad that may impregnated with buffer salts and surfactants that make the sample suitable for interaction with the detection system.

In a further embodiment, the lateral flow test strips may contain one or more target capture zones. The surface of capture zones is modified with an entity that specifically binds to an analyte of interest, for example, the microbe or microbe-based angents in the environmental sample. The modification of the surface of capture zones may be achieved by linking the solid support to, for example, antibodies, proteins, nucleotides, and/or other compounds. Such modification may be the same or different modification applied to nanocrystals. Each of the analyte capture zones may bind a different species of analyte, or may bind the same species of analyte. In lateral flow test strips where each of the analyte capture zones binds the same species of analyte, the binding may occur at varying concentrations of analyte. The capture zone can be any shape, as long as it attracts the sample and solvent flow from the sample receiving area through the analyte capture zones.

In one embodiment, the lateral flow test strips exhibit tolerance for variations in pH (e.g., pH 2-12), ion strength, viscosity, and biological matrices, contributing to few, if any, false positive and false negative results.

Up-conversion luminescence is based on the absorption of two or more low-energy (longer wavelength, typically infrared) photons by a nanocrystal followed by the emission of a single higher-energy (shorter wavelength) photon. Some aspects of lateral flow assays using UCP's have been described in Corstjens et al. (2014), Feasibility of Lateral Flow Test for Neurocysticercosis Using Novel Up-Converting Nanomaterials and a Lightweight Strip Analyzer, PloS Negl. Trop. Dis. 8(7):e2944. which is incorporated herein by reference in its entirety.

### B. PCR Assays

In another embodiment, the materials and methods of the subject invention are combined with PCR procedures to create a highly sensitive assay. The incorporation of uniform-sized nanocrystal UCPs into PCR products generated via amplification using one (or both) PCR primer(s) coupled to the nanocrystals at the 5′ end of the oligonucleotide primers provides superior assay characteristics when compared to standard reporter molecules used for detection.

Advantageously, unlike commonly used reporter molecules (e.g., alkaline phosphatse and horseraddish peroxidase), the signals produced from the nanocrystals are devoid of background florescence and lack interference with other biological molecules. In addition, because the UCP signal lasts up to 20 years, the signal can be temporally integrated to increase the sensitivity of the assay. Advantageously, the uniformity of the nanocrystal size and morphology enable stoichiometric coupling of the UCP to the oligonucleotide, which improves sensitivity, quantitation and the dynamic range of the assay.

Additionally, nanocrystal reporter pairs with complementary optical properties can be utilized in a variety of homogeneous based systems and assays designed to determine co-localization of specific target markers on a single sequence, protein, cell, etc. The complementary nanocrystal pairs exhibit unique optical properties such that, when in proximity to each other, the emission from nanocrystal A will activate nanocrystal B. In a specific example, a NaYF4:YbTm composition having a 980 nm excitation and 800 nm emission can excite a NaYF4:YbTmNd composition having an 808 nm excitation and an emission signature around 980 nm.

The optically complementary nanocrystal reporters enable the (1) identification of co-localized targets, (2) identification of specific binding events in a homogeneous mixture (without separation), and (3) multiplexed identification of the presence of markers along specific oligonucleotide sequences as well as co-localization. For assay targets where there is expected to be low target numbers, inexpensive concentration of the target species using, for example, well-known magnetic bead-based technologies can be readily implemented.

### C. Multi-well Assays

In another embodiment, the assay of the invention may be performed on multi-well arrays, for example, 8, 12, 24, 48, 96, 192, 384-well arrays, in a high-throughput setting.

### Analytes

The present invention provides methods and devices to efficiently and accurately detect, quantify and/or track microbes, microbe-based agents, and/or other analytes in environmental samples.

The analytes can be microbes, microbe-based agents and/or analytes arising from the presence or activity of microbes. The microbes can be beneficial microorganisms or pathogens, including agricultural pathogens.

Microbes that can be detected, quantified and/or tracked according to the subject invention include, but are not limited to bacteria, archaea, yeast, fungi, viruses, protozoa, and multicellular organisms. The microbe-based agents that can be analytes according to the subjection invention include, but are not limited to, composition containing microbes, microbe metabolites and other microbe growth by-products. In one embodiment, the present invention further provides methods for detecting a product produced by an entity (such as an animal or plant) in response to a microbe and/or microbe-based agent.

In one embodiment, the present invention further provides methods for detecting a product produced by an entity (such as an animal or plant) in response to a microbe and/or microbe-based agent.

In one embodiment, the method detects a product, produced by an entity infected by an agricultural pathogen. The entity can be a plant or a part of the plant including leaf, stem, root, and flower. The environmental sample may include, but is not limited to, soluble plant extracts, and insoluble plant extract.

In certain embodiments, the product produced by an entity in response to a microbe and/or microbe-based agent may be a protein, polypeptide, nucleotide and/or other molecule. The product may be secreted into the environment or food sample.

### A. Beneficial Microbes

The microbes that can be detected according to the subject invention include, but not limited to bacteria, archaea, yeast, fungi, viruses, protozoa, or multicellular organisms.

In one embodiment, the microorganisms are bacteria, including gram-positive and gram-negative bacteria. These bacteria may be, but are not limited to, for example, Escherichia coli, Rhizobium (e.g., Rhizobium japonicum, Sinorhizobium meliloti, Sinorhizobium fredii, Rhizobium leguminosarum biovar trifolii, and Rhizobium etli), Bradyrhizobium (e.g., Bradyrhizobium japanicum, and B. parasponia), Bacillus (e.g., Bacillus subtilis, Bacillus firmus, Bacillus laterosporus, Bacillus megaterium, Bacillus amyloliquifaciens), Azobacter (e.g., Azobacter vinelandii, and Azobacter chroococcum), Arhrobacter (e.g. Agrobacterium radiobacter), Pseudomonas (e.g., Pseudomonas chlororaphis subsp. aureofaciens (Kluyver)), Azospirillium (e.g., Azospirillumbrasiliensis), Azomonas, Derxia, Beijerinckia, Nocardia, Klebsiella, Clavibacter (e.g., C. xyli subsp. xyli and C. xyli subsp. cynodontis), cyanobacteria, Pantoea (e.g., Pantoea agglomerans), Sphingomonas (e.g., Sphingomonas paucimobilis), Streptomyces (e.g., Streptomyces griseochromogenes, Streptomyces qriseus, Streptomyces cacaoi, Streptomyces aureus, and Streptomyces kasugaenis), Streptoverticillium (e.g., Streptoverticillium rimofaciens), Ralslonia (e.g., Ralslonia eulropha), Rhodospirillum (e.g., Rhodospirillum rubrum), Xanthomonas (e.g., Xanthomonas campestris), Erwinia (e.g., Erwinia carotovora), Clostridium (e.g., Clostridium bravidaciens, and Clostridium malacusomae), and combinations thereof.

In certain embodiments, the methods are used to detect and/or track Bacillus subtilis in the environment. In one embodiment, the microbe comprises Bacillus subtilis strains such as, for example, B. subtilis var. lotuses strains B1 and B2, which are effective producers of surfactin.

In one embodiment, the microorganism is a fungus (including yeast), including, but not limited to, for example, Starmerella, Mycorrhiza (e.g., vesicular-arbuscular mycorrhizae (VAM), arbuscular mycorrhizae (AM)), Mortierella, Phycomyces, Blakeslea, Thraustochytrium, Penicillium, Phythium, Entomophthora, Aureobasidium pullulans, Fusarium venenalum, Aspergillus, Trichoderma (e.g., Trichoderma reesei, T. harzianum, T. viride and T. hamatum), Rhizopus spp, endophytic fungi (e.g., Piriformis indica), Saccharomyces (e.g., Saccharomyces cerevisiae, Saccharomyces boulardii sequela and Saccharomyces torula), Debaromyces, Issalchenkia, Kluyveromyces (e.g., Kluyveromyces lactis, Kluyveromyces fragilis), Pichia spp (e.g., Pichia pastoris), killer yeasts, such as Wickerhamomyces (e.g., Wickerhamomyces anomalus) and combinations thereof.

More specifically, the method can be used to detect one or more viable fungal strains capable of controlling pests, bioremediation, enhancing oil recovery and other useful purposes, e.g., Starmerella bombicola, Candida apicola, Candida batistae, Candida floricola, Candida riodocensis, Candida stellate, Candida kuoi, Candida sp. NRRL Y-27208, Rhodotorula bogoriensis sp., Wickerhamiella domericqiae, as well as any other sophorolipid-producing strains of the Starmerella clade.

In another embodiment, the microorganism is a yeast. A number of yeast species are suitable for production according to the current invention, including, but not limited to, Saccharomyces (e.g. Saccharomyces cerevisiae, Saccharomyces boulardii sequela and Saccharomyces torula), Debaromyces, Issakhenkia, Kluyveromyces (e.g. Kluyveromyces lactis, Kluyveromyces fragilis), Pichia spp (e.g. Pichia pastoris), and combinations thereof.

In certain embodiments, the microbes may be chosen from strains of killer yeast. In another embodiment, the microbes are Wickerhamomyces anomalus strains.

Wickerhamomyces anomalus, also known as Pichia anomala and Hansenula anomala, is frequently associated with food and grain production. It is capable of growing on a wide range of carbon sources at low pH, under high osmotic pressure, and with little or no oxygen, allowing for its survival in a wide range of environments.

In specific embodiments, the subject invention provides a method to detect the W. anomalus yeast strain and mutants thereof in the envrionment. Procedures for making mutants are well known in the microbiological art. For example, ultraviolet light and nitrosoguanidine are used extensively toward this end. In one embodiment, the microbe is the Starmerella yeast clade, such as Starmerella bombicola.

In one embodiment, the microorganism is an archaea, or eubacteria, including, but not limited to, Methanobacteria, Methanococci, Methanomicrobia, Methanopyri, Halobacteria, Halococci, Thermococci, Thermoplasmata, Thermoproetei, Psychrobacter, Arthrobacter, Halomonas, Pseudomonas, Hyphomonas, Sphingomonas, Archaeoglobi, Nanohaloarchaea, extremophilic archaea, such as thermophiles, halophiles, acidophiles, and psychrophiles, and combinations thereof.

In one embodiment, the microbe is a virus, including but not limited to adenovirus, cytomegalovirus, viruses of the herpes family, varicella zoster, influenza, rhinovirus, measles, mumps, enteroviruses, and the like.

In specific embodiments, microbes for the production of SLPs can be Candida sp., Cryptococcus sp., Cyberlindnera samutprakarnensis JP52 (T), Pichia anomala, Rhodotorula sp., or Wickerhamiella sp.

In further specific embodiments, microbes for the production of MELs can be Pseudozyma sp., Candida sp., Ustilago sp., Schizonella sp., or Kurtzmanomyces sp.

Other microbial strains including, for example, other microbial strains capable of digesting polymers or accumulating significant amounts of, for example, glycolipid-biosurfactants, enzymes, solvents, or other useful metabolites can also be used in accordance with the subject invention. For example, useful metabolites according to the present invention include mannoprotein, beta-glucan and other metabolites that have bio-emulsifying and surface/interfacial tension-reducing properties.

### B. Pathogens

In one embodiment, the present invention provides methods for detecting pathogens in the environmental samples. The pathogens may include, but not limited to, a member of one the genera Yersinia, Klebsiella, Providencia, Erwinia, Enterobacter, Salmonella, Serratia, Aerobacter, Escherichia, Pseudomonas, Shigella, Vibrio, Aeromonas, Streptococcus, Staphylococcus, Micrococcus, Moraxella, Bacillus, Clostridium, Corynebacterium, Eberthella, Francisella, Haemophilus, Bacteroides, Listeria, Erysipelothrix, Acinetobacter, Brucella, Pasteurella, Flavobacterium, Fusobacterium, Streptobacillus, Calymmatobacterium, Legionella, Treponema, Borrelia, Leptospira, Actinomyces, Nocardia, Rickettsia, Micrococcus, Mycobacterium, Neisseria, or Campylobacter.

The pathogens may also include, but not limited to a pathogenic virus such as, a member of the Papilloma viruses, Parvoviruses, Adenoviruses, Herpesviruses, Vaccine virus, Arenaviruses, Coronaviruses, Rhinoviruses, Respiratory syncytial viruses, Influenza viruses, Picornaviruses, Paramyxoviruses, Reoviruses, Retroviruses, Rhabdoviruses, or human immunodeficiency virus (HIV).

The pathogens may further include, but not limited to a member of one of the genera Taenia, Hymenolepsis, Diphyllobothrium, Echinococcus, Fasciolopsis, Heterophyes, Metagonimus, Clonorchis, Fasciola, Paragonimus, Schistosoma, Enterobius, Trichuris, Ascaris, Ancylostoma, Necator, Wuchereria, Brugi, Loa, Onchocerca, Dracunculus, Naegleria, Acanthamoeba, Plasmodium, Trypanosoma, Leishmania, Toxoplasma, Entamoeba, Giardia, Isospora, Cryptosporidium, Enterocytozoa, Strongyloides, or Trichinella.

According to the subject invention, the pathogens may include, but not limited to a fungus such as, for example, Ringworm, Histoplasmosis, Blastomycosis, Aspergillosis, Cryptococcosis, Sporotrichosis, Coccidiodomycosis, Paracoccidioidomycosis, Mucomycosis, Candidiasis, Dermatophytosis, Protothecosis, Pityriasis, Mycetoma, Paracoccidiodomycosis, Phaeohphomycosis, Pseudallescheriasis, Trichosporosis, or Pneumocystis.

In one embodiment, the pathogens according to the subject invention may include, but not limited to bovine papular stomatitus virus (BPSV), bovine herpes virus (BVH), bovine viral diarrhea (BVD), foot-and-mouth disease virus (FMDV), blue tongue virus (BTV), swine vesicular disease virus (SVD), porcine respiratory reproductive syndrome virus (PRRS), vesicular stomatitis virus (VSV), and vesicular exanthema of swine virus (VESV).

In specific embodiments, the pathogen according to the subject invention may be Neisseria meningitides, Streptococcus agalactiae, Staphylococcus aureus, Porphyromonas gingivalis, Chlamydia pneumoniae, Bacillus anthracis, Streptococcus suis, Echinococcus granulosus, Streptococcus sanguinis, and Helicobacter pylori.

In one embodiment, the pathogen according to the subject invention may produce toxic molecules that pose threat to human health and crop growth. For example, Aspergillus flavus and Aspergillus parasiticus produce aflatoxin B1 (AFB1), a highly toxic aflatoxin, which can contaminate grains and other crops such as peanut, corn, rice, and soybean. Other toxins produced by pathogen include, but are not limited to, ochratoxin A, botulinum toxin, shiga toxin 1, shiga toxin 2, and staphylococcal enterotoxin B.

### Plants

Plants that can be tested according to methods of the subject invention include: Row Crops (e.g., Corn, Soy, Sorghum, Peanuts, Potatoes, etc.), Field Crops (e.g., Alfalfa, Wheat, Grains, etc.), Tree Crops (e.g., Walnuts, Almonds, Pecans, Hazelnuts, Pistachios, etc.), Citrus Crops (e.g., orange, lemon, grapefruit, etc.), Fruit Crops (e.g., apples, pears, etc.), Turf Crops, Ornamentals Crops (e.g., Flowers, vines, etc.), Vegetables (e.g., tomatoes, carrots, etc.), Vine Crops (e.g., Grapes, Strawberries, Blueberries, Blackberries, etc.), Forestry (eg, pine, spruce, eucalyptus, poplar, etc), Managed Pastures (any mix of plants used to support grazing animals).

Further plants that can benefit from the products and methods of the invention include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Further examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Plants of interest further include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc.

### Plant Diseases

Examples of plant diseases that can be detected according to the present invention, include the following:

Diseases of wheat: Fusarium head blight (Fusarium graminearum, F. avenacerum, F. culmorum, Microdochium nivale), Typhula snow blight (Typhula sp., Micronectriella nivalis), loose smut (Ustilago tritici, U. nuda), bunt (Tilletia caries), leaf blotch (Mycosphaerella graminicola), and glume blotch (Leptosphaeria nodorum);

Diseases of corn: smut (Ustilago maydis) and brown spot (Cochliobolus heterostrophus); Diseases of citrus: melanose (Diaporthe citri), scab (Elsinoe fawcetti), penicillium rot (Penicillium digitatum, P. italicum), and Citrus Greening (Candidatus Liberibacter spp.);

Diseases of apple: blossom blight (Monilinia mali), powdery mildew (Podosphaera leucotricha), Alternaria leaf spot (Alternaria alternata apple pathotype), scab (Venturia inaequalis), bitter rot (Colletotrichum acutatum), and crown rot (Phytophtora cactorum);

Diseases of pear: scab (Venturia nashicola, V. pirina), black spot (Alternaria alternata Japanese pear pathotype), rust (Gymnosporangium haraeanum), and phytophthora fruit rot (Phytophtora cactorum);

Diseases of peach: brown rot (Monilinia fructicola), scab (Cladosporium carpophilum), and phomopsis rot (Phomopsis sp.);

Diseases of grape: anthracnose (Elsinoe ampelina), ripe rot (Glomerella cingulata), black rot (Guignardia bidwellii), downy mildew (Plasmopara viticola), and gray mold (Botrytis cinerea); Diseases of Japanese persimmon: anthracnose (Gloeosporium kaki) and leaf spot (Cercospora kaki, Mycosphaerella nawae);

Diseases of gourd: anthracnose (Colletotrichum lagenarium), Target leaf spot (Corynespora cassiicola), gummy stem blight (Mycosphaerella melonis), Fusarium wilt (Fusarium oxysporum), downy mildew (Pseudoperonospora cubensis), and Phytophthora rot (Phytophthora sp.); Diseases of tomato: early blight (Alternaria solani), leaf mold (Cladosporium fulvum), and late blight (Phytophthora infestans);

Diseases of cruciferous vegetables: Alternaria leaf spot (Alternaria japonica), white spot (Cercosporella brassicae), and downy mildew (Peronospora parasitica);

Diseases of rapeseed: sclerotinia rot (Sclerotinia sclerotiorum) and gray leaf spot (Alternaria brassicae);

Diseases of soybean: purple seed stain (Cercospora kikuchii), sphaceloma scad (Elsinoe glycines), pod and stem blight (Diaporthe phaseolorum var. sojae), rust (Phakopsora pachyrhizi), and brown stem rot (Phytophthora sojae);

Diseases of azuki bean: gray mold (Botrytis cinerea) and Sclerotinia rot (Sclerotinia sclerotiorum);

Diseases of kidney bean: gray mold (Botrytis cinerea), sclerotinia seed rot (Sclerotinia sclerotiorum), and kidney bean anthracnose (Colletotrichum lindemthianum);

Diseases of peanut: leaf spot (Cercospora personata), brown leaf spot (Cercospora arachidicola), and southern blight (Sclerotium rolfsii);

Diseases of potato: early blight (Alternaria solani) and late blight (Phytophthora infestans);

Diseases of cotton: Fusarium wilt (Fusarium oxysporum); Diseases of tobacco: brown spot (Alternaria longipes), anthracnose (Colletotrichum tabacum), downy mildew (Peronospora tabacina), and black shank (Phytophthora nicotianae);

Diseases of sugar beat: Cercospora leaf spot (Cercospora beticola), leaf blight (Thanatephorus cucumeris), Root rot (Thanatephorus cucumeris), and Aphanomyces root rot (Aphanidermatum cochlioides);

Diseases of rose: black spot (Diplocarpon rosae) and powdery mildew (Sphaerotheca pannosa);

Diseases of chrysanthemum and asteraceous plants: downy mildew (Bremia lactucae) and leaf blight (Septoria chrysanthemi-indici);

Diseases of various plants: diseases caused by Pythium spp. (Pythium aphanidermatum, Pythium debarianum, Pythium graminicola, Pythium irregulare, Pythium ultimum), gray mold (Botrytis cinerea), Sclerotinia rot (Sclerotinia sclerotiorum), and Damping-off (Rhizoctonia solani) caused by Rhizoctonia spp.;

Disease of Japanise radish: Alternaria leaf spot (Alternaria brassicicola);

Diseases of turfgrass: dollar spot (Sclerotinia homeocarpa), brown patch, and large patch (Rhizoctonia solani);

Disease of banana: sigatoka (Mycosphaerella fijiensis, Mycosphaerella musicola, Pseudocercospora musae); and

Seed diseases or diseases in the early stages of the growth of various plants caused by bacteria of Aspergillus genus, Penicillium genus, Fusarium genus, Tricoderma genus, Thielaviopsis genus, Rhizopus genus, Mucor genus, Phoma genus, and Diplodia genus.

The disease may be root borne, foliar, present in the vascular system of the plant or transmitted by insects and include all bacterial, viral, and fungal pathogens of plants.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for detecting a target analyte in an environmental or food sample, comprising the steps of:
contacting the sample with a plurality of nanocrystals, wherein the nanocrystals have been surface modified with an entity that specifically binds to the analyte in the sample,
separating the nanocrystals bound to the analyte in the sample from unbound nanocrystals, and
detecting the nanocrystals that bind to the analyte.

2. The method according to claim 1, wherein the nanocrystals have unique and uniform morphology, size, and/or composition, producing a unique optical signature.

3. The method, according to claim 2, wherein the unique optical signature is manifested in rise and/or decay times.

4. The method according to claim 1, wherein the nanocrystals are up-converting phosphor particles.

5. The method according to claim 1, wherein the nanocrystals comprise at least one rare earth element selected from lanthanum (La), cerium (Ce), praseodymium (Pr), neodymium (Ne), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

6. The method according to claim 1, wherein the nanocrystals have a size ranging from 4 nm to 400 nm.

7. The method, according to claim 1, wherein the nanocrystals emit light for greater than 10⁻⁸ seconds.

8. The method, according to claim 1, wherein the nanocrystals can be excited at a wavelength from 900 nm to 1000 nm.

9. The method, according to claim 8, wherein the nanocrystals are excited at a wavelength from 960 nm to 980 nm.

10. The method, according to claim 1, wherein the nanocrystals emit light at a wavelength from 400 nm to 12,000 nm.

11. The method, according to claim 1, wherein the nanocrystals are β-phase particles.

12. The method, according to claim 1, wherein the nanocrystals are combined with a second reporter selected from quantum dots, carbon nanotubes, gold particles, silver particles, and magnetic or dye-doped particles.

13. The method according to claim 1, wherein the entity that specifically binds to the analyte is an antibody, protein, aptamer polypeptide, or polynucleotide.

14. The method, according to claim 1, wherein genomic analysis is used to identify a specific epitope from genetic sequence information of an unculturable microbe or a mixed population of microorganisms, and wherein a binding agent to the genetically-identified epitope is produced that specifically binds to the unculturable microbe.

15. The method, according to claim 1, wherein the analyte is a bacterium, yeast, fungus, or virus.

16. The method, according to claim 1, wherein the analyte is an agricultural pathogen.

17. The method, according to claim 16, wherein the agricultural pathogen is selected from pathogens that cause citrus greening disease, potato late blight, grape powdery mildew, red blotch, tobacco mosaic virus, fire blight and/or Pierce's Disease.

18. The method according to claim 1, wherein the sample is soil or plant material.

19. The method according to claim 18, wherein the sample is plant tissue.

20. The method according to claim 1, wherein the analyte is a microbe-based agent.

21. The method according to claim 20, wherein the microbe-based agent is a microbial biosurfactant or a mycotoxin.

22. The method, according to claim 1, wherein the sample is food and the analyte is a mycotoxin.

23. The method, according to claim 1, wherein the sample is a biological sample from an animal.

24. The method, according to claim 23, wherein the biological sample is a blood, fecal, mucous, saliva, or tissue sample.

25. The method, according to claim 1, wherein the sample is a water sample.

26. The method, according to claim 25, wherein the water sample is selected from drinking water, ground water, surface water and wastewater.

27. The method, according to claim 1, wherein the sample is a commercial product that contains microbes.

28. The method, according to claim 27, wherein the product is for use in agriculture.

29. The method, according to claim 27, wherein the product is a food product,

30. The method, according to claim 29, wherein the microbes are probiotics.

31. The method, according to claim 29, wherein the microbes are pathogenic.

32. The method according to claim 1, wherein the analyte is a microbe and the detection sensitivity for the analyte is 10¹ CFU/mL or less.

33. The method, according to claim 1, wherein the nanocrystals are tuned to avoid background interference from naturally occurring chromophores in a sample.

34. The method, according to claim 1, wherein multiple independently-tuned nanocrystals are placed in a multiplexed array on a single support to facilitate analysis of multiple analytes from a single sample.

35. The method, according to claim 1, wherein 5 or more analytes are analyzed simultaneously

36. The method, according to claim 1, wherein the method is performed within 100 yards of where the sample was obtained.

37. The method, according to claim 1, wherein the method is performed within 10 minutes of when the sample was obtained.

38. The method according to claim 1, wherein the detecting step is performed in a single readout.

39. (canceled)

40. The method, according to claim 1, wherein the detection, quantification and/or tracking of the analyte is done by a farmer, regulatory official, compliance official, or distributer.

41. The method, according to claim 1, where the assay is conducted at any point in the supply chain from immediately post-production of a commercial product to just prior to use of the product.

42. The method, according to claim 1, wherein data from individual tests are transmitted to a database that can be accessed from a location that is remote from the location where the test was performed.

43. The method, according to claim 42, wherein the data is used to assess performance of beneficial microbes or assess the movement of pathogens.

44. The method according to claim 1, which is accomplished using a lateral flow or microfluidic assay.

45. The method according to claim 44, wherein the lateral flow or microfluidic assay is an immunoassay.

46. The method according to claim 44, wherein the assay is performed using a portable detection device.

47. The method, according to claim 46, wherein the portable detection device comprises an LED and a camera,

48. The method, according to claim 47, wherein the portable detection device is a cell phone.

49. The method according to claim 44, wherein the assay is carried out utilizing a multiple flow technique.

50. The method according to claim 44, wherein a lateral flow test strip has a solid support comprising one or more sample receiving areas and one or more target capture zones.

51. The method according to claim 50, wherein the solid support is nitrocellulose or engineered microfluidic channels etched or molded into a plastic or glass substrate.

52. The method according to claim 48, wherein the target capture zone has been surface modified to specifically bind microbes or microbe-based agents in the environmental sample.

53. A device for performing the assay of claim 1.

54. The device, according to claim 53, comprising nanocrystals that have been surface modified with an entity that specifically binds to the target analyte.

55. The device, according to claim 54, wherein the nanocrystals have unique and uniform morphology, size, and/or composition, producing a unique optical signature.

56. The device, according to claims 55, wherein the unique optical signature is manifested in rise and/or decay times.

57. The device, according to claim 54, wherein the nanocrystals are up-converting phosphor particles.

58. The device, according to claim 54, wherein the nanocrystals comprise at least one rare earth element selected from lanthanum (La), cerium (Ce), praseodymium (Pr), neodymium (Ne), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

59. The device, according to claim 54, wherein the nanocrystals have a size ranging from 4 nm to 400 nm.

60. The device, according to claim 54, wherein the nanocrystals emit light for greater than 10⁻⁸ seconds.

61. The device, according to claim 54, wherein the nanocrystals can be excited at a wavelength from 900 nm to 1000 nm.

62. The device, according to claim 61, wherein the nanocrystals are excited at a wavelength from 960 nm to 980 nm.

63. The device, according to claim 54, wherein the nanocrystals emit light at a wavelength from 400 nm to 12,000 nm.

64. The device, according to claim 54, wherein the nanocrystals are β-phase particles.

65. The device, according to claim 54, wherein the nanocrystals are combined with a second reporter selected from quantum dots, carbon nanotubes, gold particles, silver particles, and magnetic or dye-doped particles.

66. The device, according to claim 54, wherein the entity that specifically binds to the analyte is an antibody, protein, aptamer polypeptide, or polynucleotide.

67. The device, according to claim 54, wherein the nanocrystals are tuned to avoid background interference from naturally occurring chromophores in a sample.

68. The device, according to claim 54, wherein multiple independently-tuned nanocrystals are placed in a multiplexed array on a single support to facilitate analysis of multiple analytes from a single sample.

69. The device, according to claim 54, wherein said device can transmit data from individual tests to a database that can be accessed from a location that is remote from the location where the test was performed.

70. The device, according to claim 54, which is a lateral flow or microfluidic assay.

71. The device, according to claim 70, wherein the lateral flow or microfluidic assay is an immunoassay.

72. The device, according to claim 54, comprising, as one component of the device, a portable detection unit.

73. The device, according to claim 72, wherein the portable detection unit comprises an LED and a camera.

74. The device, according to claim 73, wherein the portable detection unit is a cell phone.

75. The device, according to claim 54, wherein the assay is carried out utilizing a multiple flow technique.

76. The device, according to claim 54, wherein a lateral flow test strip has a solid support comprising one or more sample receiving areas and one or more target capture zones.

77. The device, according to claim 76, wherein the solid support is nitrocellulose or engineered microfluidic channels etched or molded into a plastic or glass substrate.

78. The device, according to claim 76, wherein the target capture zone has been surface modified to specifically bind microbes or microbe-based agents in the environmental sample.

79. An assay for detecting a target polynucleotide sequence using PCR, wherein said method comprises the use of primer sequences to amplify said target polynucleotide sequence wherein at least one of said primer sequences is coupled to a nanocrystal.
